(19) United States
(12) Patent Application Publication
(10) Pub. No.: US 2014/0171619 A1
(43) Pub. Date: Jun. 19, 2014

(54) PEPTIDE-CROSSLINKED BIOACTIVE POLYMERIC MATERIALS

(71) Applicants: Matthew Becker, Stow, OH (US); Matthew Graham, Akron, OH (US); Frank Harris, Boca Raton, FL (US); Fei Lin, Akron, OH (US)

(72) Inventors: Matthew Becker, Stow, OH (US); Matthew Graham, Akron, OH (US); Frank Harris, Boca Raton, FL (US); Fei Lin, Akron, OH (US)

(73) Assignee: The University of Akron, Akron, OH (US)

(21) Appl. No.: 14/122,903

(22) PCT Filed: May 25, 2012
(86) PCT No.: PCT/US2012/039578

§ 371 Date: Mar. 3, 2014

Related U.S. Application Data
(60) Provisional application No. 61/490,990, filed on May 27, 2011.

(51) Int. Cl. C07K 7/06

(52) U.S. Cl. CPC C07K 7/06
USPC 530/329

(57) ABSTRACT

A method for creating a peptide crosslinked bioactive polymeric material includes reacting a hydroxy-functionalized small molecule with a amino acid to form an amino acid functionalized monomer, reacting the amino acid functionalized monomer with a urea bond former to form a amino acid-based poly(ester urea), and reacting the amino acid-based poly(ester urea) with a peptide based crosslinker to form the peptide crosslinked bioactive polymeric material.

### FIELD OF THE INVENTION

The present invention generally relates to bioactive polymeric materials. In particular embodiments the present invention relates to bioactive polymeric materials for regenerative medical applications in vivo. In other embodiments, the present invention relates to peptide-crosslinked polymeric materials for use in vivo and, in particular embodiments, to peptide-crosslinked amino acid-based poly(ester urea) (PEU) materials providing bioactivity in vivo. In other embodiments, the present invention provides peptide crosslinked amino acid PEU materials that provide osteoinductive activity. In other embodiments, the present invention provides a specific scaffold structure formed from peptide crosslinked amino acid PEU materials.

### BACKGROUND OF THE INVENTION

Synthetic, degradable polymers have been used in a myriad of ways for regenerative medicine and orthopaedic applications. However, they generally lack the mechanical properties necessary for load-bearing surgical interventions. Numerous examples are found in the literature where degradable polymers have been used successfully in orthopaedic applications, including poly(lactic acid), polycaprolactone and poly(propylene fumarate); however, the maximum reported mechanical properties for these polymers are within the range of 3.0-3.5 GPa. For comparison, the elastic modulus of cortical bone within the mid-diaphysis of a long bone, along the axis of the bone is approximately 18 GPa. It is generally accepted that poly(lactic acid) has insufficient mechanical properties to sustain load-bearing applications. There is a need in the art for resorbable biomaterials that possess high moduli for use in regenerative medicine and orthopaedic applications.

Ideally the mechanical properties of a scaffold must be appropriate to regenerate bone in load bearing sites. It is unlikely that stand-alone polymers will reach those numbers. Researchers have increased the mechanical properties of degradable materials through composite and blending approaches, yet there still remains a challenge to engineer polymeric materials with sufficient mechanical properties that retain the ability to fully degrade fully. Traditional methods to mechanically reinforce the polymers, including covalent crosslinking, generally limit or prevent the desired biodegradation. One strategy has been to use naturally occurring amino acids as building blocks for monomer precursors. However, conventional poly(a-amino acids), despite their biological origin, possess distinct physical, chemical and biodegradation properties that limit their synthetic utility. However, poly(ester urea) materials described herein are a significant step in the right direction in that they are both strong and degradable.

Significant limitations in bringing new materials to the clinic include the findings that fully synthetic materials lack cell specific receptors and have poorly defined serum adsorption properties, which can vary widely depending on the amount and nature of the adsorbed layer. Recent advances in synthetic polymer chemistry have enabled the synthesis of polymeric materials designed to elicit specific cellular functions and to direct cell-cell interactions. For example, polymers have been derivatized with adhesive receptor-binding peptides, glycoproteins and tethered growth factors to enhance interactions at the biological-synthetic interface. Other proposed solutions include doping polymers with proteins or peptides or decorating the polymer with covalently tethered peptides that mimic an extracellular matrix or growth factors. One of these examples is osteogenic growth peptide (OGP). OGP is a naturally occurring 14-mer peptide growth factor found in serum at μmol/L concentrations. As a soluble peptide, OGP regulates proliferation, differentiation, and matrix mineralization in osteoblast lineage cells. The active portion of OGP, the OGP(10-14) region, is cleaved from the peptide and binds to the OGP receptor which activates multiple signaling pathways including the MAP kinase, the Src, and the RhoA pathways. When administered intravenously to animals, OGP and OGP(10-14) promote increased bone density and stimulate healing, which suggests a potential use in bone tissue engineering applications.

Despite advances made, there is a need in the art for new classes of polymeric materials useful in orthopedic and other regenerative medicine applications, materials that impart specific osteogenic signaling motifs and have suitable mechanical properties for their target application.

### SUMMARY OF THE INVENTION

In one embodiment, the present invention provides a peptide based crosslinker according to the following formula:

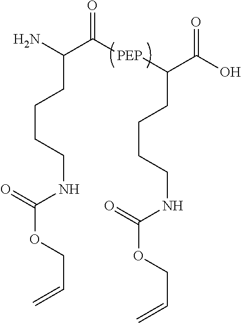

wherein PEP is a peptide with 20 or less amino acids.

In other embodiments, the present invention provides a peptide based crosslinker according to paragraph [0006] in which PEP is a peptide selected from the group consisting of bone sialoprotein, vitronectin, fibronectin, osteogenic growth peptide, and bone morphogenetic protein-2.

In other embodiments, the present invention provides a method for creating a peptide crosslinked bioactive polymeric material comprising the steps of:
a. reacting a hydroxy-functionalized small molecule with an amino acid to form an amino acid functionalized monomer,
b. reacting the amino acid functionalized monomer with a urea bond former to form a amino acid-based poly(ester urea),
c. and reacting the amino acid-based poly(ester urea) with a peptide based crosslinker to form the peptide crosslinked bioactive polymeric material, wherein the peptide based crosslinker has the following structure:

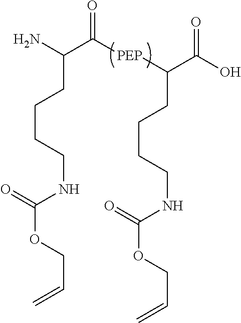

wherein PEP is a peptide with 20 or less amino acids.

In other embodiments, the present invention provides a method as in paragraph [0008], in which the hydroxy-functionalized small molecule is any organic molecule of less than twenty carbons and having at least two hydroxy-end groups.

In other embodiments, the present invention provides a method as in paragraphs [0008] or [0009], in which the hydroxy-functionalized small monomer is a hyrodxy-functionalized diol or triol.

In other embodiments, the present invention provides a method as in any of paragraphs [0008] through [0010], in which the hydroxy-functionalized small monomer is 1,6-hexanediol.

In other embodiments, the present invention provides a method as in any of paragraphs [0008] through [0011], in which the amino acid has the following structure:

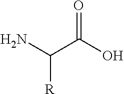

wherein R is

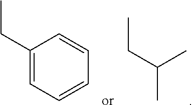

In other embodiments, the present invention provides a method as in any of paragraphs [0008] through [0012] in which the peptide based crosslinker has the following structure:

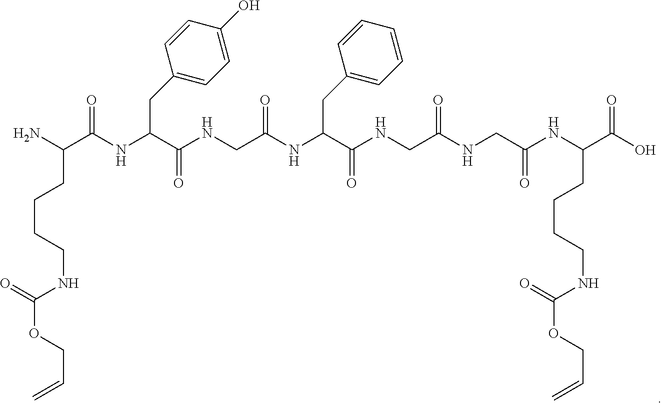 [SMILES: C=CCOC(=O)NCCCCC(N)C(=O)NC(Cc1ccc(O)cc1)C(=O)NCC(=O)NC(Cc1ccccc1)C(=O)NCC(=O)NCC(=O)NC(CCCCNC(=O)OCC=C)C(=O)O]

In other embodiments, the present invention provides a method as in any of paragraphs [0008] through [0013], in which the amino acid is any amino acid other than serine.

In other embodiments, the present invention provides a method as in any of paragraphs [0008] through [0014], in which the urea bond former is phosgene or triphosgene.

In other embodiments, the present invention provides a method as in any of paragraphs [0008] through [0015], in which PEP is a member selected from the group consisting of bone sialoprotein, vitronectin, fibronectin, osteogenic growth peptide, and bone morphogenetic protein-2.

### BRIEF DESCRIPTION OF THE DRAWINGS

FIG. 1 is a general reaction scheme for the production of peptide-crosslinked amino acid-based poly(ester urea);

FIG. 2 provides graphs of Instron testing to measure yield strength (YS) and tensile strength (TS) of poly(1-PHE-6) and poly(1-LEU-6);

FIG. 3 provides WST-1 proliferation assay of MC3t3-E1 osteoblast and primary murine fibroblast cells;

FIG. 4 shows digital images of histology stained slides stained with Masson's Tribrome at 100 times magnification; and

FIG. 5 shows graphs of quantitative histological analysis of the respective measurements as collected from Masson's Tricrome analysis at 4 weeks and 12 weeks.

### DETAILED DESCRIPTION OF ILLUSTRATIVE EMBODIMENTS

The present invention provides peptide crosslinked mechanically robust and bioactive polymeric materials. In other embodiments, the present invention provides peptide crosslinked amino acid-based poly(ester urea) (PEU) materials that can be used for useful applications. In other embodiments, the present invention provides peptide crosslinked amino acid PEU materials that provide osteoinductive activity. In other embodiments, the present invention provides a specific scaffold structure formed from peptide crosslinked amino acid PEU materials.

The present invention provides a particular reaction scheme that is suitable for forming the peptide crosslinked polymeric materials of this invention. A hydroxy-functionalized small molecule, typically either a diol or triol, is reacted with an amino acid, which results in end functionalizing the molecule with an amino acid based material, forming what is termed herein an amino acid functionalized monomer. A urea bond is then introduced into the amino acid end functionalized monomer using either triphosgene diphosgene or phosgene to form a poly(ester urea) (PEU). The PEU is then crosslinked with a peptide-based crosslinker to form the peptide crosslinked amino acid-based PEU material.

The hydroxy-functionalized small monomer may be selected from virtually any organic molecule of less than twenty carbons and having at least two hydroxy-end groups. In other embodiments, hydroxy functionalized compound may possess between 3 and 8 hydroxy functional groups. These groups may arise from sugar molecules, carbohydrates, and branched diols.

In particular embodiments, the molecule chosen is hydroxy-end functionalized hexane, 1,6 hexanediol.

The amino acid may be selected from virtually any amino acid, with the proviso that serine is not suitable because of the hydroxyl present on the side chain.

The reaction of the hydroxyl-functionalized molecule with the amino acid to create an amino acid functionalized monomer can be achieved in any number of ways generally known to those of skill in the art. Generally, a condensation reaction at temperatures exceeding the boiling point of water involving a slight molar excess (˜2.1 eq.) of the acid relative to the hydoxy groups is sufficient to enable the reaction to proceed. The presence of toluene sulphonic acid is necessary to protonate the amine on the amino acid and ensure that trans amidation reactions do not occur at higher conversions.

To introduce the urea bond to the resultant amino acid functionalized monomer, phosgene, diphosgene or triphosgene is employed. Diphosgene (a liquid) and triphosgene (a solid crystal) may be found more suitable than phosgene because they are generally appreciated as safer substitutes to phosgene, which is a toxic gas.

The reaction of an amino acid functionalized monomer with triphosgene, diphosgene or phosgene to create an amino acid-based PEU can also be achieved in any number of ways generally known to those of skill in the art. Generally, a large molar excess (˜10-50 molar excess relative to the amine concentration) is required to drive the reaction to higher molecular masses.

A peptide crosslinker is employed to crosslink the amino acid-based PEU. The peptide crosslinker may be selected from those with the general structure shown below:

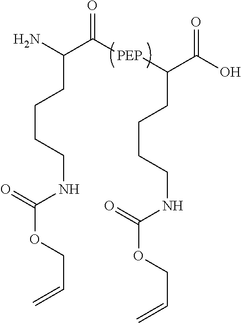

wherein PEP is selected from virtually any peptide with 20 or less amino acids, and having a desired bioactivity (osteoconductive, osteoinductive, adhesive, anti-inflammatory, angiogenic, neurostimulatory). It will be appreciated that a lysine group (K) is bonded to each end of the PEP. In a particular embodiment, PEP is chosen from the group consisting of bone sialoprotein (KRSR, sequence GGGKRSR), vitronectin, fibronectin (RGD, sequence GRGDS), osteogenic growth peptide (OGP, sequence ALKRQGRTLYGFGG), an osteogenic growth peptide subunit (OGP[10-14], sequence YGFGG) and bone morphogenetic protein-2 (BMP-2, sequence KIPKASSVPTELSAISTLYL). Notably, the peptide (PEP) is end functionalized on both ends with lysine (K).

The peptide crosslinker is generated by placing lysine amino acid residues at both the N-terminus and C-terminus of the target peptide (i.e. PEP) during the solid phase synthesis approach. The lysine side chain amino acids are prederivatized with Aloc units during the solid phase synthesis approach. Prior to cleavage from the resin, the N terminus of the growing peptide is acetylated. This yields a functional peptide based crosslinker with precisely two vinyl groups (one at each end).

The amino acid-based PEU is reacted with the peptide crosslinker to create a peptide crosslinked amino acid-based PEU. The peptide crosslinker can be incorporated between 0.1 to 5.0 mole % without decreasing the mechanical properties of the base PEU polymer. The exact location of the crosslinkers within the composite cannot be determined experimentally. During the peptide crosslinking process, radical formation in situ is undoubtedly leading to chain scission in the polymer backbone. While one would generally expect rapid loss of mechanical properties, this does not happen in this instance due to low mole fraction of crosslinker and high initial molecular mass.

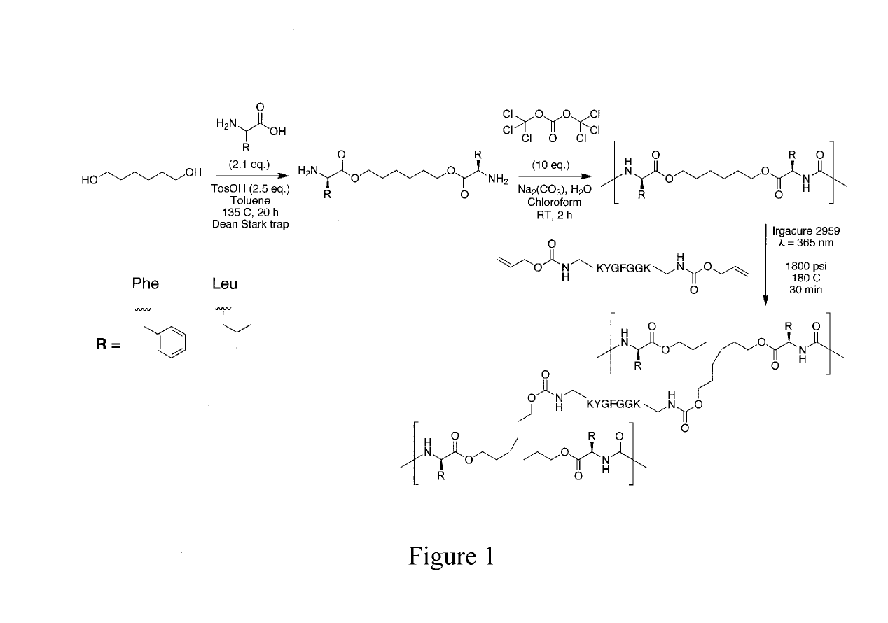

The general reaction scheme for particular embodiments in accordance with this invention is shown in FIG. 1. It will be appreciated that the peptide-based crosslinker formula has been simplified by use of “K” in place of the majority of the lysine structure. The crosslinker specifically has the following structure:

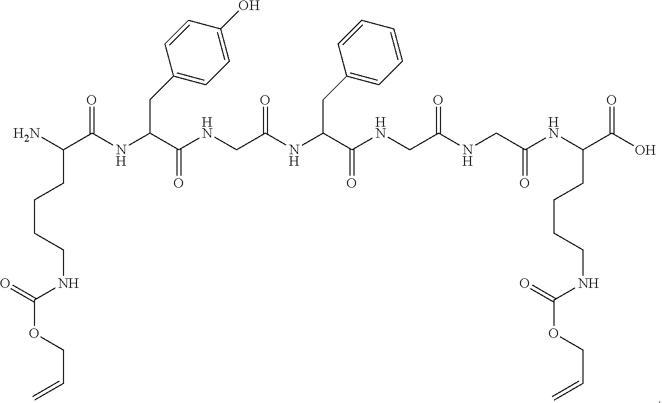 [SMILES: C=CCOC(=O)NCCCCC(N)C(=O)NC(Cc1ccc(O)cc1)C(=O)NCC(=O)NC(Cc1ccccc1)C(=O)NCC(=O)NCC(=O)NC(CCCCNC(=O)OCC=C)C(=O)O]

In a specific embodiment, the diol is 1,6-hexanediol. The diol is reacted with 2.1 equivalents of an amino acid as shown in FIG. 1, by mixing with toluene sulfonic acid (TosOH 2.5 equivalents) and toluene at 135° C. for 20 hours. The amino acid replaces the hydroxyl groups of the diol with the amino acid to provide an amino-acid functionalized monomer as shown. Urea bonds are then introduced to this amino acid functionalized monomer by reaction with triphosgene, mixed with sodium carbonate, water and chloroform. This creates an amino acid-based PEU, as shown in FIG. 1.

Next, the amino acid-based PEU is crosslinked with a peptide-based crosslinker having a desired peptide that is modified with lysine groups at each end. The desired peptide employed is osteogenic growth peptide[10-14] (OGP[10-14] is a subunit of ALKRQGRTLYGFGG). The amino-acid based PEU is mixed with the peptide-based crosslinker, photoinitiator Irgacure 2959 and hexafluoride-2-propanol. The resulting crosslinked polymer can then be crushed into small pieces and melt pressed at a pressure of from about 1200 psi to 1800 psi and a temperature of from about 130° C. to 180° C. to form a desired scaffold. The resulting peptide crosslinked amino acid-based PEU is shown in FIG. 1.

The peptide crosslinked amino acid-based PEU of this embodiment has the advantage of having mechanical properties (Elastic modulus=6.1 GPa) that are nearly twice that of poly(lactic acid) (elastic modulus=2.9 GPa). The general biocompatibility and resorption of the peptide crosslinked amino acid-based PEU polymers where shown in an in vivo rat subcutaneous experiment. While not a clinical orthopaedic model, it is an important step to demonstrate the utility of this new class of biomaterials. The peptide crosslinked amino acid-based PEU's have been shown to promote integration between the polymer construct and host.

The crosslinked amino acid-based PEU's of this invention may be employed to create scaffolds, porous scaffolds, fibers, webbing and mesh.

In light of the foregoing, it should be appreciated that the present invention significantly advances the art by providing a peptide crosslinked amino acid-based poly(ester urea) that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

The present invention describes the efforts to develop a new class of crosslinked, mechanically-robust polymeric materials for orthopedic applications. The methods include enhanced mechanical properties in addition to imparting specific osteogenic signaling motifs. To mechanically reinforce the polymers and stimulate specific biological activity this invention incorporates OGP based crosslinkers. Peptide-crosslinked phenylalanine and leucine-based poly(ester urea) (PEU) homopolymers were synthesized and tethered with 0.5% and 1.0% OGP(10-14). In addition, the semi-crystalline nature of poly(ester urea)s afford non-chemical methods in which the mechanical properties, chemical stability, and biodegradation rates can be tailored. This example describes in detail the chemical, mechanical, in vitro and in vivo data which demonstrate enhanced moduli, biocompatibility and resorption of the poly(ester urea) materials. Further the data herein highlights the many opportunities that the community will find for these materials in regenerative medicine applications.

### Materials

Unless listed otherwise, all solvents and reagents were purchased from Sigma-Aldrich and used as received. Fluorenylmethyloxycarbonyl (FMOC) protected amino acids and preloaded Wang-resins were purchased from CEM Corp. Alpha minimum essential medium (α-MEM) and ultra culture media were purchased from Lonza. All other cell culture reagents were purchased from Invitrogen Corp. All reagents were used as received.

### Synthesis of Di-p-toluenesulfonic Acid Salts of Bis-L-phenylamine and Bis-L-Leucine Esters

Di-p-toluene sulfonic acid salts of Bis-L-phenylamine and Bis-L-leucine esters were prepared using procedures as discussed in Pang X et al., Synthesis, Characterization and Biodegradation of Functionalized Amino Acid-based Bioanalogous Polymers., Biomaterials 2010; 31:3745, which is incorporated herein by reference, as shown in FIG. 1. Briefly, L-Leucine (1.31 g, 10 mmol), 1,6-hexanediol (0.48 g, 4 mmol), p-toluenesulfonic acid (1.92 g, 10 mmol), and toluene (20 mL) were mixed in a 250 mL 3-neck flask equipped with Dean Stark trap and a magnetic stir bar. The system was purged with nitrogen for 30 min after which the reaction mixture was heated at 135° C. under nitrogen for 20 h. The reaction mixture was allowed to cool to ambient temperature and the crude product was isolated by vacuum filtration. The organic residue was recrystallized four times using 25 mL water to yield 2.26 g (82%) of compound 1 di-p-toluenesulfonic acid salt of Bis-L-leucine ester as a white power. The product was characterized with ¹H-NMR, ¹³C-NMR, and melting point measurements.

Di-p-toluenesulfonic acid salt of Bis-L-leucine hexane-1,6-diester (Monomer 1, 1-LEU-6): mp: 186-188° C.; ¹H-NMR (300 MHz, DMSO): 0.90 (d, 12H) 1.34 (s, 4H) 1.45-1.80 (m, 8H) 2.29 (s, 6H) 3.99 (t, 2H) 4.15 (d, 4H) 7.13 (d, 4H) 7.49 (d, 4H) 8.31 (s, active H); ¹³C-NMR (75 MHz, DMSO): 169.91, 145.34, 137.35, 129.10, 125.48, 65.52, 50.62, 27.76, 24.75, 23.79, 23.13, 21.92, 20.79.

Di-p-toluenesulfonic acid salt of Bis-L-phenylalanine hexane-1,6-diester (Monomer 2, 1-PHE-6): mp: 215-217° C.; ¹H-NMR (300 MHz, DMSO): 0.90-1.15 (m, 4H) 1.38 (s, 4H) 1.25-1.50 (m, 4H) 2.23 (s, 6H) 2.91-3.09 (m, 2H) 3.10-3.21 (m, 2H) 4.01 (t, 4H) 4.30 (t, 2H) 7.11 (d, 4 H) 7.19-7.40 (m, 10H) 7.49 ((d, 4H) 8.43 (s, active H); ¹³C-NMR (75 MHz, DMSO): 169.06, 145.00, 138.12, 134.70, 129.32, 128.55, 128.21, 127.54, 125.53, 65.45, 53.34, 36.20, 27.63, 24.70, 20.82.

### Interfacial Polycondensation of 1-LEU-6 and 1-PHE-6

A general scheme for poly (ester urea) synthesis is given in FIG. 1. Monomer 1-LEU-6 (6.89 g, 10 mmol), sodium carbonate (3.18 g, 30 mmol), and water (150 mL) were mixed in 500 mL 3-neck flask equipped with an overhead mechanical stirrer and a thermometer. The mixture was heated with a warm water bath at 40° C. for 30 min. The water bath was removed and replaced with ice-salt bath. When inside temperature decreased to about 0° C., pre-prepared triphosgene solution (1.035 g, 3.30 mmol in 30 mL chloroform)) was added to the reaction system quickly (5 seconds) with fast mechanical stirring. The reaction was allowed to proceed for 30 min and then additional aliquots of triphosgene (0.108 g, 0.330 mmol, total) were dissolved in chloroform (5 mL) and 1 mL aliquots were added into the reaction system every 10 min. After the addition of the triphosgene, the organic phase was precipitated into hot water, filtered, and dried in vacuum to yield a white solid (3.2 g, 74.5% yield). The product was characterized by ¹H-NMR, ¹³C-NMR, FT-IR spectroscopy, SEC, TGA, and DSC. Molecular weight and thermal properties of polymers are listed in Table 1.

Poly (1-LEU-6): FT-IR (cm⁻¹): 1740 [—C(CO)—O—], 1648, 1542 [—NH'C(O)—NH—], 3283 [—NH—C(O)—NH—]; ¹H-NMR (300 MHz, DMSO): 0.91 (d, 12H) 1.20-2.00 (m, 14H) 4.21 (t, 4H) 4.45 (d, 2H) 5.35-5.80 (m, active H); ¹³C-NMR (75 MHz, DMSO): 174.64, 157.08, 65.00, 51.60, 65.00, 51.00, 42.31, 28.20, 25.29, 24.74, 22.61, 22.12.

Poly (1-PHE-6): FT-IR (cm⁻¹): 1736 [—C(CO)—O—], 1649, 1553 [—NH—C(O)—NH—], 3384 [—NH—C(O)—NH—]; ¹H-NMR (300 MHz, DMSO): 0.91 (d, 12H) 1.20-2.00 (m, 14H) 4.21 (t, 4H) 4.45 (d, 2H) 5.35-5.80 (m, active H); ¹³C-NMR (75 MHz, DMSO): 174.64, 157.08, 65.00, 51.60, 65.00, 51.00, 42.31, 28.20, 25.29, 24.74, 22.61, 22.12.

TABLE 1
Characterization Data Summary for the Amino Acid-Based PEU
TABLE 1. CHARACTERIZATION DATA SUMMARY FOR
THE AMINO ACID-BASED POLY(ESTER UREAS)

| SAMPLES | M_{w} | M_{w}/Mₙ | T_{g} | Tₘ | Td | G′ (GPa) |
|---|---|---|---|---|---|---|
| Poly(1-LEU-6) | 76,800 | 2.12 | 57 | 126 | 275 | 4.4 ± 0.9 |
| Poly(1-PHE-6) | 84,000 | 2.42 | 107 | 153 | 335 | 6.1 ± 1.1 |

### Peptide Crosslinker

A symmetric vinyl functionalized OGP [10-14] was synthesized using the (Aloc)KYGFGGK(Aloc) sequence by solid phase FMOC chemistry. Peptides were cleaved from resin using standard conditions (45 min, 95% trifluoroacetic acid (TFA), 2.5% triisopropylsilane (TIPS), 2.5% water (by volume)) and precipitated in cold diethyl ether. Following two trituration cycles, the peptides were dialyzed in deionized water (molecular weight (MW) cutoff 100 g/mol, cellulose membrane, Pierce), and molecular weight was verified with matrix assisted laser desorption ionization time of flight (MALDI-TOF) (FW (+H) 924.50 g/mol, required 924.44 g/mol).

### Molecular Mass Characterization

Number-average (Mₙ) and weight-average (M_{w}) molecular weights and molecular weight distribution (M_{w}/Mₙ) were determined by size exclusion chromatography (SEC). The instrument was equipped a guard column and set of 50 Å, 100 Å, 104 Å, and linear (50-104 Å) Styragel 5 μm columns, a Waters 486 tunable UV/vis detector, and a Waters 410 differential refractometer. All the analyses were carried out with a flow rate of 1 mL/min using RI detector. DMF was used as the eluent at 50° C. The respective molecular weights and molecular weight distributions of each polymer were determined using a universal calibration curve, which was obtained by plotting ln-([η]/Mn) (which is the natural log of the intrinsic viscosity divided by the number average molecular weight) as a function of elution volume, after calibration with polystyrene standards (Polymer Laboratories).

### Thermal Characterization

The degradation temperatures (T_{d}) of poly(1-LEU-6) and poly(1-PHE-6) materials were determined by thermogravimetric analysis (TA instruments, Q50 TGA) across a temperature range of 30° C. to 500° C. at a scanning rate of 20° C./min under nitrogen. The thermal transitions of the poly(1-LEU-6) and poly(1-PHE-6) materials were characterized by differential scanning calorimetry (TA Instruments, Q2000 DSC) at a scanning rate of 10° C./min.

### Crosslinked Scaffold Fabrication

Polymer plugs were prepared by a compression molding fabrication process. Polymer materials ˜1 g, and a corresponding amount of peptide crosslinker and photoinitiator Irgacure 2959 were dissolved in 20 mL hexafluoride-2-propanol. The clear solution was photo irradiated with 365 nm UV light for 45 minutes. The solvent was evaporated in a fume hood at room temperature for 24 h followed by vacuum drying at 80° C. for 24 h. The composite was crushed into small pieces and melt pressed into sheets using a Carver Hydraulic unit model 3912 with pressure 1800 psi at designed temperature (130° C. for poly(1-LEU-6) and 180° C. for poly(1-PHE-6)). The polymeric block was cooled to room temperature, and annealed in vacuum at proper temperature for 24 h (80° C. for poly(1-LEU-1) and 130° C. for poly(1-PHE-6)). The final polymeric block was cut into small circular plugs with diameters 0.5 cm for testing. All plugs were stored in nitrogen atmosphere at temperature −20° C. for future use.

### Mechanical Characterization

Dynamic Mechanical Analysis (DMA): The Young's moduli of the poly(1-PHE-6), 0.5% OGP poly(1-PHE-6), and 1.0% OGP poly(1-PHE-6) data were determined using a TA Q800 dynamic mechanical analysis (DMA) instrument with sample dimensions 40×2.0×0.2 mm at ambient temperature. The stain rate was 1.5%/sec. Using small strains (<0.15%) the Young's moduli were determined using the slope of the tangent line in the linear regime. Stress strain data were reported using the TA Universal Analysis software. The data were plotted in Origin 8 and Young's modulus values were calculated using regression analysis in the linear regime. Values for Young's moduli and standard deviations were determined from four individual measurements.

Instron: The elastic modulus and tensile properties of the poly(1-LEU-6) and the poly(1-PHE-6) were measured using an Instron 3365 universal materials testing machine. The gauge length was 20 mm and the crosshead speed was set at 30 mm/min. The specimens were 40 mm long, 4 mm wide and 0.2 mm thick. Stress strain data were reported using the Instron Bluehill software. The data were plotted in Origin 8 and elastic modulus values were calculated using regression analysis in the linear regime prior to the yield point. Results presented are average values for six individual measurements. The elastic modulus was calculated using the slope of the tangent line of the data curve prior to the yield point.

### In Vitro Cell Culture and Characterization

Primary human foreskin fibroblasts were obtained from stock cultures isolated from infant male circumcision tissue specimens maintained at the Calhoun Research Laboratory, Akron General Medical Center. MC3T3 E1 osteoblasts were obtained from Riken. Fibroblasts were maintained in DMEM with high glucose (Gibco, 11965) and MC3T3-E1 osteoblasts in MEM Alpha (Gibco A10490; Invitrogen, Carlsbad, Calif.). Each media was supplemented with 10% Fetal Bovine Serum (FBS) (Sigma, F6178) (St. Louis, Mo.) and 1% Penicillin-Streptomcycin-Fungizone (10,000 U:10,000 μg:25 μg) (Lonza BioWhittaker, BW17-745E; Fisher Scientific, Waltham, Mass.). Cells were maintained in an incubator at 37° C. and 5% carbon dioxide: 95% air. In preparation for morphologic and proliferation experiments, cells were released from the flasks using 2.5% trypsin ethylenediamine tetraacetic acid (EDTA) (Invitrogen, 15090-046) and counted on a hemacytometer for viability and concentration determination using 0.4% Trypan Blue (Gibco, 15250). A preliminary morphological assessment was performed with plugs of the poly(1-PHE-6) and poly(1-LEU-6) polymers tethered with 1.0% OGP in the presence of fibroblast cells at a concentration of 10⁵ cells/ml in 6-well plates. After 48 hours, cells were viewed with an Olympus CKX41 inverted microscope (Center Valley, Pa.) and digital images were captured using QImaging software and a Micropublisher Real Time Viewing (RTV) 5.0 charge-coupled device (CCD) color cooled camera (Qlmaging, Princeton, N.J.).

Cellular proliferation was measured using a WST-1 viability assay (Dojindo Molecular Technologies, W201-10; Rockville, Md.). Briefly, a plugs of PLA, poly(1-PHE-6) or poly(1-LEU-6) base polymer and tethered with 0.5% or 1.0% OGP was sterilized by rinsing in isopropyl alcohol and applied to 6-well plates containing 4×10⁵ fibroblast or MC3T3 cells. Following incubation for 48 hours, the polymers were transferred to a 96-well plate with care taken not to disrupt cells on polymer surfaces and rinsed in Tyrode's Hepes buffer. The WST-1 assay solution was added, incubated with the polymers for 2 hours, the resultant reaction solution was transferred to clean wells and absorbance was read at 450 nm.

### Animal Surgeries

All animal protocols regarding the handling, care, maintenance and surgical procedures were reviewed and approved by the Institutional Animal Care and Use Committee of Akron General Medical Center. A total of 16 male Sprague Dawley rats (Harlan Laboratories, Indianapolis, Ind.) weighing >250 grams were divided into groups containing 8 animals each per base polymer type, poly(1-PHE-6) or poly(1-LEU-6). All animals were pre-anesthetized by a subcutaneous injection with 10-12 mg/kg of butorphanol mixed with 0.04 mg/kg of atropine. Following inhalant anesthesia induction at 3% isofluorane in 100% oxygen, the animal was maintained at 1-1.5% isoflluorane in 100% for the duration of the surgical procedures.

On the dorsum, four incisions were created with a sterile scalpel blade, two in the left and right lateral direction approximately 1 cm from the spine and approximately 2 cm apart. A subcutaneous pocket was tunneled using hemostats in the posterioanterior direction. Thin plugs of polymers sterilized by ethylene oxide were inserted into each pocket and skin incisions were closed with Michel clips. Within the four subcutaneous spaces, each animal received: (1) PLA; (2) either poly(1-LEU-6) or poly(1-PHE-6); and, poly(1-LEU-6) or poly(1-PHE-6) tethered with either (3) 0.5% or (4) 1.0% OGP. Positions 1-4 were randomized for each animal, while retaining diagonal distribution of control and test materials, to account for variability in positioning on the back. After 4 or 12 weeks post-surgical insertion, four animals from each group were euthanized and tissues containing the polymers were collected (2 cm×2 cm), preserved in formaldehyde and prepared for histological evaluation.

### Histology and Histomorphometric

Tissue sections (5 μm) were cut (Leica RM2235 micrometer) and stained by hematoxylin and eosin (Ventana ST5020 Automated Stainer, Hematoxylin 7211 and Eosin 71204) to show normal tissue architecture, Mallory's trichrome (Ventana NEXES Special Stains, Trichrome II Staining Kit 860-013) for collagen deposition and cellular infiltration, and Alizarin red to detect mineralization of calcium as evidence of bone cell activity. For Alizarin Red S staining, histological sections stained with 40 mM Alizarin Red S solution, pH 4.2 at ambient temperature for 10 min, rinsed five times in distilled water and washed for 15 min in 1× PBS. Histological sections were counter-stained with hematoxylin, dehydrated with ethanol and rinsed in xylene before mounting with permamount. All slides were examined with an Olympus BX51 light microscope to identify the location of the polymer within the subcutaneous region and digital images were captured using the QImaging camera and software. Histomorphometric features were analyzed on the digital images of the Trichrome sections at 40× using BioQuant Nova Prime Image Analysis software (v.6.75.10; Nashville, Tenn.). The total area (μm²) of connective tissue surrounding and including the polymer was delineated as the region of interest (ROI). A separate area measurement (μm²) was defined for the regions of tissue extending from the connective tissue capsule into the region containing the polymer, indicating polymer degradation. Within the ROI, the threshold was selected to identify the regions of red stain and a video count array measured the total pixel area (μm²) of collagen deposition/cellular infiltration. The percentages of degradation and cellular infiltration were calculated in relation to the respective total area ROI. The width of the connective tissue capsule was measured at 5 random locations surrounding the polymer and averaged. The numbers of giant cells and blood vessels were counted within and adjacent to the ROI.

### Statistics

Histomorpometric data for comparisons of the polymer constructs were statistically evaluated using one-way analysis of variance (ANOVA) and linear discriminate correlation. Mean values and standard errors are reported, unless otherwise noted. T-tests were performed to identify individual comparative differences. Statistical analysis for all other measurements was performed using one-way analysis of variance (ANOVA) and a comparison-wise Tukey test at 95% confidence. Mean values and standard deviation are reported, unless otherwise noted. The standard deviations of the mean were used as an estimate for the standard uncertainty associated with each measurement technique.

### Synthesis and Characterization

Katsarava et al., Amino Acid-based Bioanalogous polymers., Synthesis and Study of Regular Poly(ester amide)s based on Bis(alpha-amino acid) Alpha, Omega-alkyne Diesters, and Aliphatic Dicarboxylic Acids., J Polym Sci Part A: Polym Chem 1998;37:391, which is incorporated herein by reference, discussed a synthesis of homo-PEUs, without using diisocyanates, via active polycondensation. In this procedure, active carbonates (e.g. di-p-nitrophenyl carbonate) interact with di-p-toluenesulfonic acid salts of bis(α-amino acid)-α,ω-alkylene diesters. The molecular weight can be tailored by altering the molecular weights of the constituent monomers and the degree of polymerization. This experiment used a modified version of the process for the synthesis of the base PEU materials which are described as x-amino acid-y where x and y are the number of carbon atoms in the chain as shown in FIG. 1. The leucine (LEU) and phenylalanine (PHE) amino acid-based PEUs utilized 1,6-hexane diol, and are denoted 1-LEU-6 and 1-PHE-6. The molecular weight, molecular weight distribution and thermal properties of the poly(1-LEU-6) and poly(1-PHE-6) were measured (Table 1). At ambient temperature, poly(1-PHE-6) is not soluble in conventional organic solvents, but is soluble in hexafluoroisopropanol and 3:1 mixtures of tetrachloroethane:phenol. When the poly(1-LEU-6) was melt processed, the Tg remained the same but no melting peak is observed indicating that no crystallinity was present. This indicates that polymer crystallinity can be suppressed using the appropriate processing method. The degradation temperatures (Td) of the poly(1-LEU-6) and poly(1-PHE-6) materials were over 100° C. higher than the melting temperature indicating that both materials can be melt processed with limited impact of thermal degradation. These characteristics afford processing techniques such as molding and melt processing to be used to fabricate our scaffolds.

### Mechanical Properties

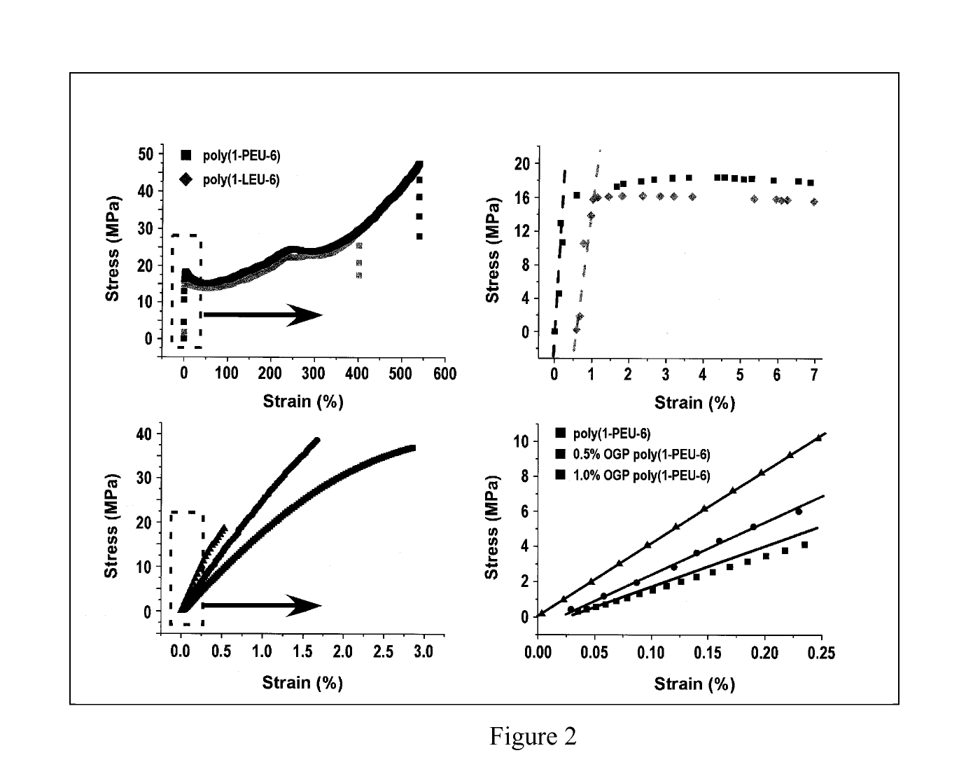

The mechanical properties of the PEU plugs where characterized by Instron and Dymanic Mechanical Analysis methods reported in Table 2, which characteristically report the Young's and elastic modulus, respectively. The Instron data show clearly that values for both the poly(1-LEU-6) and poly(1-PHE-6) (4.4 and 6.1 GPa, respectively) exceed the published values for poly(L-lactic acid) (PLLA, 2.9 GPa) and poly(e-caprolactone) (PCL, 280 MPa). To maximize accuracy and provide additional information, Instron testing was used to measure the yield strength (YS) and tensile strength (TS). Poly(1-LEU-6) and poly(1-PHE-6) had TS values of ˜470% and 510% respectively (FIG. 2). The elastic modulus and tensile properties of the poly(1-LEU-6) and the poly(1-PHE-6) were measured using an Instron 3365 universal materials testing machine. The gauge length was 20 mm and the crosshead speed was set at 30 mm/min. The specimens were 40 mm long, 4 mm wide and 0.2 mm thick. Results presented are average values for six individual measurements. The elastic modulus was calculated using the slope of the tangent line of the data curve prior to the yield point. The Young's moduli of the poly(1-PHE-6), 0.5% OGP poly(1-PHE-6), and 1.0% OGP poly(1-PHE-6) data were determined using a TA Q800 dynamic mechanical analysis (DMA) instrument with sample dimensions 40×2.0×0.2 mm at ambient temperature (approx 23° C.). The strain rate was 1.5% per second. Using small strains (<0.15%) the Young's moduli were determined using the slope of the tangent line in the linear regime. Values for Young's moduli and standard deviations were determined from four individual measurements.

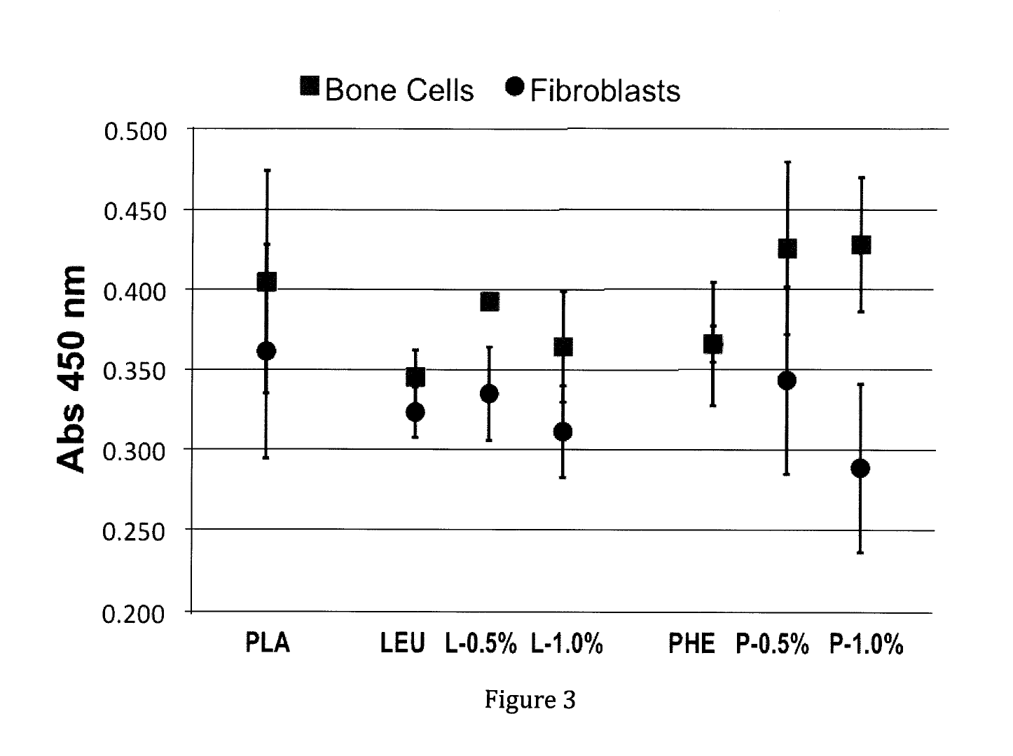

The absolute value of TS does not depend on the size of the test specimen which affords the ability extrapolate our results to larger constructs. However, it is influenced by other factors, including sample preparation, defects and temperature. Tensile strength is the opposite of compressive strength and the values can be quite different. DMA data yielded value of 3.05±0.24 GPa for the elastic modulus of poly(1-PHE-6) and showed that the elastic modulus increased proportionally with increased levels of OGP crosslinking (Table 2). The linear regime of the stress strain curve (FIG. 3) was used to calculate the Young's modulus of the poly(1-PHE-6) homopolymer as well as the 0.5% and 1.0% OGP crosslinked materials. While unoptimized, these data are significantly stronger than degradable polymers currently available clinically.

TABLE 2
Summary of Mechanical Properties of Peptide-Crosslinked PEU
TABLE 2. SUMMARY OF MECHANICAL PROPERTIES OF
PEPTIDE-CROSSLINKED POLY(ESTER UREA)

| | | | | Dynamic Mechanical Analysis | | | | |
|---|---|---|---|---|---|---|---|---|
| | Instron Testing | | | Young's Modulus | | | | |
| | Elastic Modulus | Tensile Strain | Tensile | Trial 1 | Trial 2 | Trial 3 | Trial 4 | Average |
| SAMPLES | (GPa) | (%) | Stress | (GPa) | (GPa) | (GPa) | (GPa) | (Std Dev) |
| Poly(1-LEU-6) | 4.4 ± 0.9 | 470 ± 50 | 43 ± 9 | — | — | — | — | — |
| Poly(1-PHE-6) | 6.1 ± 1.1 | 510 ± 30 | 45 ± 3 | 3.38 | 3.04 | 2.94 | 2.82 | 3.05 ± 0.24 |
| Poly(1-PHE-6) 0.5% OGP | — | — | — | 3.50 | 3.39 | 3.37 | 3.40 | 3.41 ± 0.06 |
| Poly(1-PHE-6) 1.0% OGP | — | — | — | 4.30 | 4.27 | 4.00 | 4.10 | 4.18 ± 0.14 |

### In Vitro Proliferation and Biocompatibility

Initial in vitro screening for biocompatibility and biodegradation was performed with primary human foreskin fibroblast cells and MC3T3-E1 osteoblasts. The fibroblast morphology showed an adherent state (image not shown) following cell seeding in the presence of poly(1-LEU-6) and poly(1-PHE-6) tethered with 1.0% OGP[10-14]. The cells did not display features of cytotoxicity or apoptosis. WST-1 proliferation assays were performed to confirm cellular viability and proliferative rates with poly(1-LEU-6) or poly(1-PHE-6) and polymers each tethered with 0.5% and 1.0% OGP, respectively (FIG. 3). Consistent with the biphasic, concentration-dependent proliferative effect noted in Greenberg Z., et al., Structural and Functional-Characterization of Osteogenic Growth Peptide from Human Serum—Identity with Rat and Mouse Homologs, J. Clin. Endocrinol. Metlab 1995, 80:2330, the unfunctionalized homopolymers (PLLA, poly(1-LEU-6) or poly(1-PHE-6)) do not exhibit cell-type dependent proliferative activity. However, the increasing proliferative trends in both the 0.5% and 1.0% OGP functionalized materials for the osteoblasts relative to the corresponding decreasing trends in the fibroblasts show that the peptide is bioavailable to the receptor and that we are in a bioactive concentration regime. Comparisons by two-way ANOVA of poly(lactic acid) (PLA), poly(1-LEU-6) and poly(1-PHE-6) controls showed no significant differences in fibroblast proliferation. Osteoblast proliferation was increased significantly for poly (1-LEU-6) with 0.5% OGP and for poly(1-PHE-6) with 1.0% OGP compared to polymer controls (*P<0.05). Thus, it appears that in addition to providing the mechanical reinforcement, the crosslinked OGP peptide is bioavailable to the cell surface receptors and active for signal initiation.

### In Vitro Biocompatibility and Degradation by Histological Image Analysis

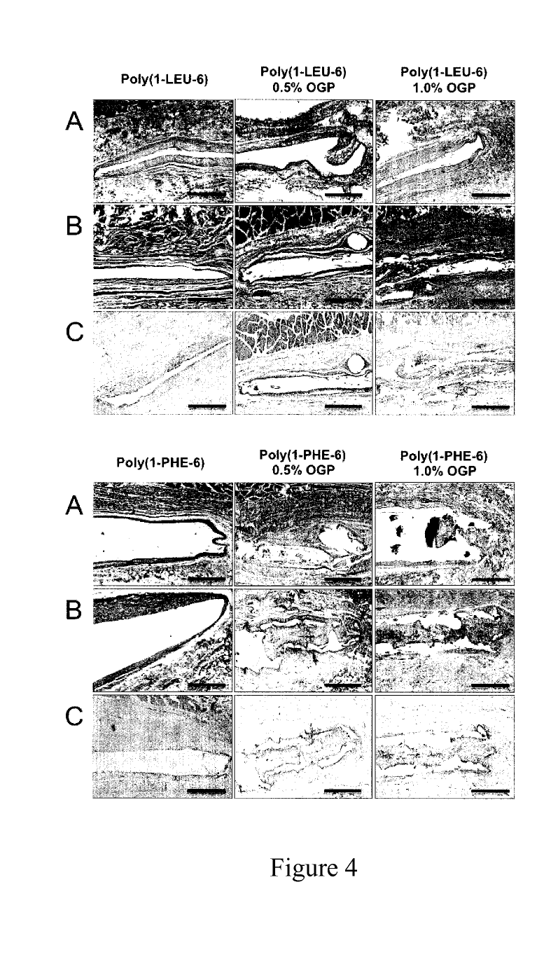

After demonstrating no appreciable effects on cellular viability, a subcutaneous implant plug model was used to assess biodegradation, cellular infiltration, capsule thickness, inflammatory response and the number of blood vessels. During necropsy for tissue collection, no evidence of fibrosis, granulomatous reactions, necrosis or bacterial infection was observed. After 4 weeks implantation, little evidence is seen in the histological sections for the degradation of either the poly(1-LEU-6) and poly(1-PHE-6) homopolymers (FIG. 4—Tissues were removed at 4 weeks (Row A) and 12 weeks (Row B) post implantation. Row C, Serial sections of 12 week histology slides shown in Row B stained with Alazirin red). The degradation rates for both of the homopolymers are not statistically different that what was measured in the PLLA control (FIG. 6A). However when crosslinked with OGP, both polymer sets show significant levels of degradation which propagate further at 12 weeks. This increase in degradation is attributed to additional free volume in the polymer plugs imparted by the OGP crosslinker and increased water uptake into the bulk material due to the peptides.

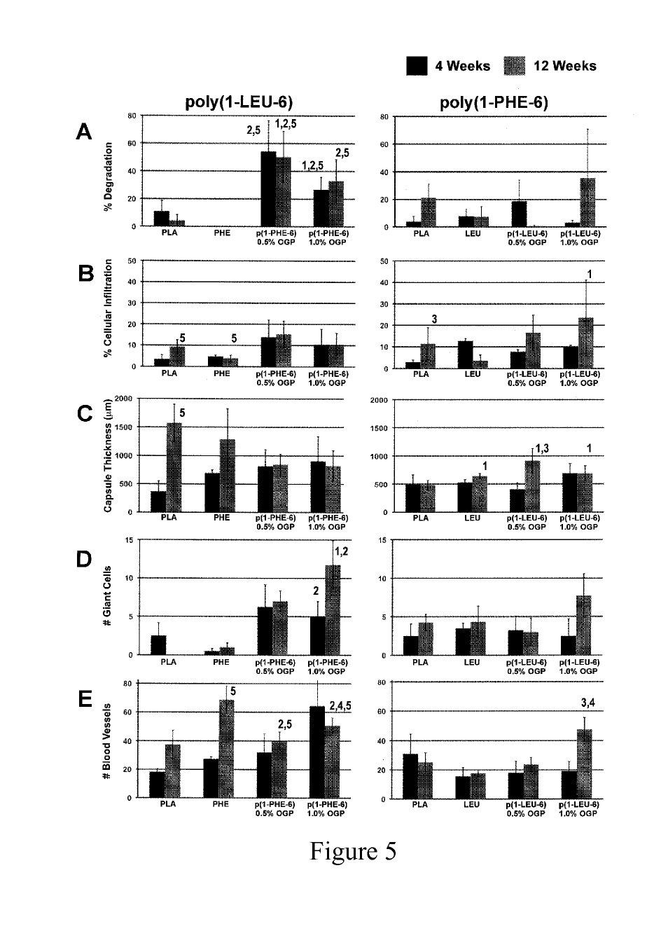

FIG. 5 provides an analysis representative of biodegradation (A) by measuring area of tissue migration within the polymer spac, cellular infiltration (B), capsule thickness (C), immune response (D) by number of giant cells present and vascularization (E) by counting the number of associated blood vessels. Statistical significance (P<0.05) was indicated by 1=compared to PLA; 2=compared to base polymer; 3=4 week versus 12 week results; 4=0.05% OGP versus 1.0% OGP; and 5=poly(1-PHE-6) versus poly(1-LEU-6). The percentage of cellular infiltration is quantitated as a pixel count of the red trichrome stain within the ROI containing the implant in the tissue section. The red stain included the nuclei of cells (predominantly lymphocytes, macrophages and fibroblasts) and the presence of collagen. The amounts of cellular infiltration in the respective data sets showed little significance other than a suggestive trend at 12 weeks in the 1% OGP crosslinked poly(1-LEU-6) relative to PLLA (FIG. 5B). The relatively low percentages of cellular migration and collagen production within the regions indicate that there were minimal inflammatory and fibrotic responses to the implanted materials.

Promising trends were seen with regard to capsule formation in both data sets (FIG. 5C). While the overall thickness data at 4 weeks were similar for PLLA, poly(1-LEU-6) and poly(1-PHE-6) homopolymers, the capsule thickness of the OGP crosslinked poly(1-PHE-6) materials did not increase appreciably while the poly(1-PHE-6) containing 0.5% and 1.0% OGP showed less capsule formation at 12 weeks relative to PLLA. The generally small capsule thicknesses (all <500 um) confirms the lack of any significant fibrotic or granulomatous responses.

The numbers of giant cells were counted as an indicator of foreign body tissue response. Giant cell counts were similar for PLLA and poly(1-LEU-6), however the 1.0% OGP crosslinked poly(1-PHE-6) materials did induce increased numbers of giant cells relative to controls (FIG. 5D). This may suggest 1% OGP is too high of a peptide concentration, yet even though the giant cell numbers were greater, the overall numbers were all comparatively small (all <20). The indication of calcium mineralization via the Alazirin red stain (FIG. 5, Row C) was not expected to be found due to the subcutaneous implantation site, absence of porosity and stem cell source, and follow-up time frame.

Biodegradation was complemented by a higher number of blood vessels in the poly(1-PHE-6) groups (FIG. 5E) compared to other test groups. An effect on vascularity has not been reported previously in the OGP literature. Since the increased blood vessel formation was not accompanied by a significant increase in inflammatory cell infiltration, it appears that recruitment and integration of blood vessels may facilitate biodegradation of the polymers.

The in vivo biocompatibility studies confirmed the in vitro results by demonstrating no adverse response from the integral tissues in contact with the polymeric materials. Capsule formation around the polymers was similar to what is normally observed for PLA. In comparison to poly(1-LEU-6), the poly(1-PHE-6) polymers with 0.5% and 1.0% OGP exhibited a significantly favorable interaction with the amount of biodegradation and incorporation of tissue into the polymer materials compared to other test groups.

What is claimed is:

1. A peptide based crosslinker according to the following formula 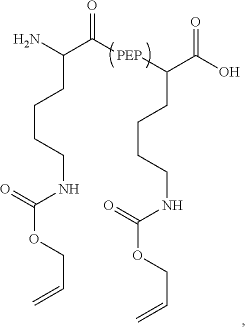
wherein PEP is a peptide with 20 or less amino acids.

2. The peptide based crosslinker according to claim 1 in which PEP is a peptide selected from the group consisting of bone sialoprotein, vitronectin, fibronectin, osteogenic growth peptide, and bone morphogenetic protein-2.

3. A method for creating a peptide crosslinked bioactive polymeric material comprising the steps of:
a. reacting a hydroxy-functionalized small molecule with a amino acid to form an amino acid functionalized monomer,
b. reacting the amino acid functionalized monomer with a urea bond former to form a amino acid-based poly(ester urea),
c. and reacting the amino acid-based poly(ester urea) with a peptide based crosslinker to form the peptide crosslinked bioactive polymeric material, wherein the peptide based crosslinker has the following structure, 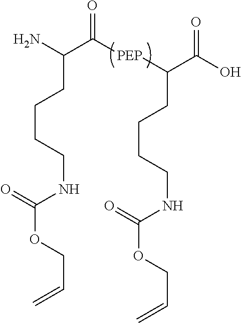
wherein PEP is a peptide with 20 or less amino acids.

4. The method of claim 3 in which the hydroxy-functionalized small molecule is any organic molecule of less than twenty carbons and having at least two hydroxy-end groups.

5. The method of claim 4 in which the hydroxy-functionalized small monomer is a hyrodxy-functionalized diol or triol.

6. The method of claim 5 in which the hydroxy-functionalized small monomer is 1,6-hexanediol.

7. The method of claim 6 in which the amino acid has the following structure 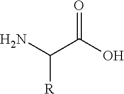
wherein R is

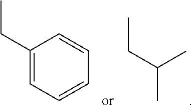

8. The method of claim 6 in which the peptide based crosslinker has the following structure:

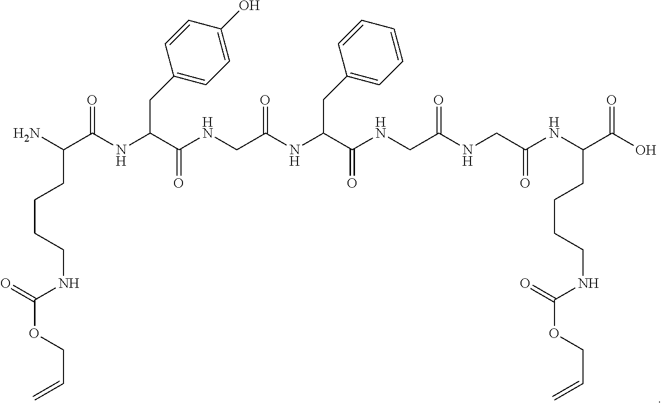 [SMILES: C=CCOC(=O)NCCCCC(N)C(=O)NC(Cc1ccc(O)cc1)C(=O)NCC(=O)NC(Cc1ccccc1)C(=O)NCC(=O)NCC(=O)NC(CCCCNC(=O)OCC=C)C(=O)O]

9. The method of claim 3 in which the amino acid is any amino acid other than serine.

10. The method of claim 3 in which the urea bond former is phosgene or triphosgene.

11. The method of claim 9 in which PEP is a member selected from the group consisting of bone sialoprotein, vitronectin, fibronectin, osteogenic growth peptide, and bone morphogenetic protein-2.
